(19) United States
(12) Patent Application Publication
(10) Pub. No.: US 2025/0253019 A1
(43) Pub. Date: Aug. 7, 2025

(54) MEDICAL IMAGE ANALYZING APPARATUS, MEDICAL IMAGE ANALYZING METHOD, AND MEDICAL INFORMATION PROCESSING APPARATUS

(71) Applicant: CANON MEDICAL SYSTEMS CORPORATION, Otawara-shi (JP)

(72) Inventor: Minoru NAKATSUGAWA, Yokohama (JP)

(73) Assignee: CANON MEDICAL SYSTEMS CORPORATION, Otawara-shi (JP)

(21) Appl. No.: 19/003,413

(22) Filed: Dec. 27, 2024

(30) Foreign Application Priority Data
Jan. 4, 2024 (JP) 2024-000191

(51) Int. Cl. G16H 10/60; G16H 30/40; G16H 50/70

(52) U.S. Cl. CPC G16H 10/60; G16H 30/40; G16H 50/70

(57) ABSTRACT

A medical image analyzing apparatus according to an embodiment includes processing circuitry. The processing circuitry is configured to obtain a medical image of a target patient and information indicating an area of interest in the medical image as accompanying information, access a patient information database storing medical images of a plurality of patients, make an evaluation of a similarity measure for the patients based on image analysis of the obtained accompanying information and the medical images of the patients, and extract similar patient similar to the target patient using the evaluation, obtain a plurality of medical images and medical documents created for the extracted similar patients, obtain a summary of the medical images by analyzing the medical images, and create a document regarding the medical image of the target patient based on the summary of the medical images, the obtained accompanying information, and a summary of the obtained medical documents.

### CROSS REFERENCE TO RELATED APPLICATIONS

This application is based upon and claims the benefit of priority from the prior Japanese Patent Application No. 2024-191, filed on Jan. 4, 2024, the entire contents of which are incorporated herein by reference.

### FIELD

Embodiments disclosed in the present specification and the drawings relate to a medical image analyzing apparatus, a medical image analyzing method, and a medical information processing apparatus.

### BACKGROUND

When an interpretation report of a medical image is created, the interpretation report might be created with reference to past reports of similar cases in the past. It is difficult, however, to identify similar cases from a huge amount of past case data and create a report with similar content.

For example, a technique for searching for similar interpretation reports in the past from an anatomical position of a key image and local structure information in the vicinity thereof is known. A technique for extracting a keyword from an interpretation report and searching for similar cases having similar diagnosis flows is also known. In these techniques, however, similar cases are identified on the basis of information regarding either an image or an interpretation report, and there is a possibility that appropriate similar cases for creating an interpretation report are not identified. In addition, an AI technology for learning data regarding hundreds of millions of combinations of images and explanatory texts and generating an explanatory sentence from an image is known. For example, OpenAI's CLIPCAP combines an image classification model CLIP and a large language model GPT to generate an explanatory sentence from an image. With such a technology, however, it is difficult to collect large-scale data in the medical field, and there is a possibility that sufficient accuracy is not achieved for medical images. It is therefore desired to support creation of a convenient and appropriate interpretation report.

### BRIEF DESCRIPTION OF THE DRAWINGS

FIG. 1 is a block diagram illustrating a configuration example of a medical information processing apparatus according to an embodiment;

FIG. 2 is a flowchart illustrating an operation example of the medical information processing apparatus according to the embodiment;

FIG. 3 is a flowchart illustrating a step of extracting similar patients in the operation example of the medical information processing apparatus according to the embodiment;

FIG. 4 is a flowchart illustrating a step of creating a medical report of a target patient in the operation example of the medical information processing apparatus according to the embodiment;

FIG. 5 is a diagram illustrating a process of obtaining medical reports of similar patient in the operation example of the medical information processing apparatus according to the embodiment;

FIG. 6 is a diagram illustrating the step of creating a medical report of a target patient in the operation example of the medical information processing apparatus according to the embodiment;

FIG. 7 is a flowchart illustrating operation of a medical information processing apparatus according to a first modification of the embodiment;

FIG. 8 is a diagram illustrating a step of displaying a medical report of a target patient and medical reports of a plurality of similar patients in the operation of the medical information processing apparatus according to the first modification of the embodiment;

FIG. 9 is a flowchart illustrating operation of a medical information processing apparatus according to a second modification of the embodiment;

FIG. 10 is a diagram illustrating a step of displaying a medical report of a target patient and medical reports of a plurality of similar patients in the operation of the medical information processing apparatus according to the second modification of the embodiment;

FIG. 11 is a diagram illustrating a step of displaying a medical report of a target patient and medical reports of a plurality of similar patients in operation of a medical information processing apparatus according to a third modification of the embodiment;

FIG. 12 is a diagram illustrating a step of displaying a medical report of a target patient and medical reports of a plurality of similar patients in operation of a medical information processing apparatus according to a fourth modification of the embodiment; and

FIG. 13 is a flowchart illustrating a step of obtaining medical reports of similar patients in operation of a medical information processing apparatus according to a fifth modification of the embodiment.

### DETAILED DESCRIPTION

A medical information processing apparatus according to an embodiment will be described in detail hereinafter with reference to the drawings. In the following description, components having substantially the same functions and configurations will be given the same reference numerals, and redundant description thereof will be made only when necessary.

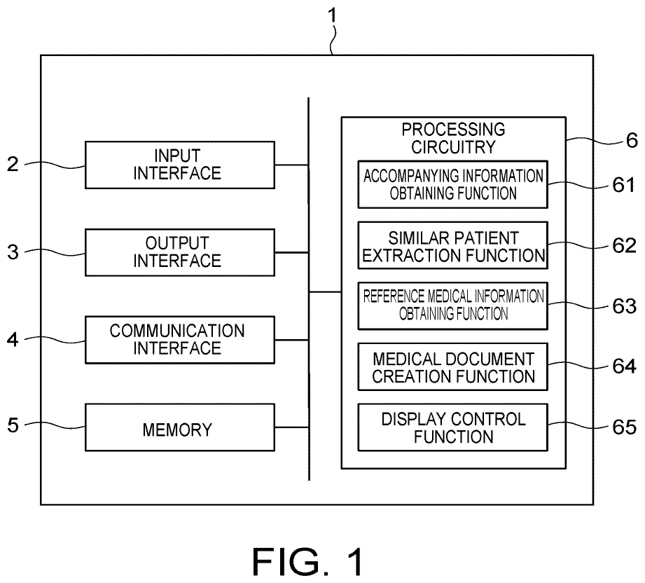

FIG. 1 is a block diagram illustrating a configuration example of a medical information processing apparatus 1 (i.e., a medical image analyzing apparatus) according to an embodiment. As illustrated in FIG. 1, the medical information processing apparatus 1 includes an input interface 2, an output interface 3, a communication interface 4, memory 5, and processing circuitry 6. The input interface 2, the output interface 3, the communication interface 4, and the memory 5 are communicably connected to the processing circuitry 6.

The input interface 2 receives various input operations from an operator. The input interface 2 converts a received input operation into an electrical signal and outputs the electrical signal to the processing circuitry 6. For example, the input interface 2 receives an input operation for creating a medical document of a target patient (i.e., a document regarding a medical image of the target patient) from the operator. As the input interface 2, for example, a mouse, a keyboard, a trackball, a switch, a button, a joystick, a touch pad, a touch panel display, or the like can be appropriately used.

The output interface 3 outputs various types of information. For example, the output interface 3 includes a display. The display outputs a graphical user interface (GUI) or the like for receiving a medical document generated by the processing circuitry 6 and various operations from the operator. For example, a liquid crystal display (LCD), a cathode-ray tube (CRT) display, an organic electro luminescence display (OELD), a plasma display, or any other display can be appropriately used as the display. The output interface 3 may include a speaker.

The communication interface 4 is an interface that communicably connects the medical information processing apparatus 1 to an electronic medical record system or a radiology information system (RIS). The electronic medical record system may be included in a hospital information system (HIS). The communication interface 4 includes, for example, a connector adapted to a parallel connection specification or a serial connection specification. The communication interface 4 performs, for example, communication control according to each of various standards and connects to a network to connect the medical information processing apparatus 1 to the electronic medical record system or the RIS over the network.

The memory 5 is a non-transitory storage device that stores various types of information, and is, for example, a hard disk drive (HDD), an optical disc, a solid-state drive (SSD), an integrated circuit storage device, or the like. The memory 5 stores, for example, a control program for controlling the medical information processing apparatus 1 and various types of data used to execute the control program. The memory 5 may be a drive device that reads and writes various types of information from and to a portable storage medium such as a compact disc (CD), a digital versatile disc (DVD), or a flash memory, a semiconductor memory device such as a random-access memory (RAM), or the like, in addition to the HDD, the SSD, and the like. Note that the memory 5 need not necessarily be implemented by a single storage device. For example, the memory 5 may be achieved by a plurality of storage devices, instead.

The processing circuitry 6 is circuitry that controls entire operation of the medical information processing apparatus 1 in accordance with an electrical signal of an input operation input from the input interface 2. The processing circuitry 6 includes, for example, an accompanying information obtaining function 61, a similar patient extraction function 62, a reference medical information obtaining function 63, a medical document creation function 64, and a display control function 65. The accompanying information obtaining function 61 is an example of an accompanying information obtainer. The similar patient extraction function 62 is an example of a similar patient extractor. The reference medical information obtaining function 63 is an example of a medical document obtainer. The medical document creation function 64 is an example of a medical document creator. The display control function 65 is an example of a display controller.

Here, for example, each of processing functions executed as the accompanying information obtaining function 61, the similar patient extraction function 62, the reference medical information obtaining function 63, the medical document creation function 64, and the display control function 65, which are the components of the processing circuitry 6 illustrated in FIG. 1, is stored in the memory 5 in the form of a program executable by a computer. The processing circuitry 6 is, for example, a processor. The processor constituting the processing circuitry 6 reads each program from the memory 5 and executes the program to implement the function corresponding to the read program. In other words, the processing circuitry 5 that has read each program has the corresponding function illustrated in the processing circuitry 6 of FIG. 1.

Note that FIG. 1 illustrates a case where the single processing circuitry 6 achieves the accompanying information obtaining function 61, the similar patient extraction function 62, the reference medical information obtaining function 63, the medical document creation function 64, and the display control function 65, but the embodiment is not limited to this. For example, the processing circuitry 6 may be configured by combining a plurality of independent processors together, and each processor may achieve one of the processing functions by executing the corresponding program. In addition, each processing function of the processing circuitry 6 may be achieved while being appropriately distributed or integrated into one or a plurality of processing circuits.

The accompanying information obtaining function 61 obtains accompanying information regarding the target patient. The accompanying information includes, for example, at least one of an examination purpose, a patient attribute, a medical image, information regarding an image area of interest, and an examination finding. The accompanying information obtaining function 61 obtains, for example, accompanying information received through communication with the electronic medical record system or the RIS via the communication interface 4. The accompanying information obtaining function 61 may read and obtain, from the memory 5, the accompanying information received from the electronic medical record system or the RIS and stored in the memory 5. Alternatively, the accompanying information obtaining function 61 may obtain accompanying information input by the operator via the input interface 2. For example, the accompanying information obtaining function 61 may obtain input information as accompanying information when the operator inputs a fragmentary image area of interest or a keyword in a finding for the image area with respect to a medical image obtained via the communication interface 4.

The accompanying information obtaining function 61 may obtain the medical image of the target patient and information indicating an area of interest in the medical image as the accompanying information.

The similar patient extraction function 62 extracts similar patients, who are similar to the target patient on the basis of the accompanying information obtained by the accompanying information obtaining function 61. For example, the similar patient extraction function 62 extracts a plurality of similar patients similar to the target patient. The similar patient extraction function 62 may compare the accompanying information regarding the target patient with a plurality of medical documents created for each of a plurality of past patients for each of a plurality of items, and calculate a distance between the accompanying information and the plurality of medical documents for each item. The similar patient extraction function 62 may then calculate a degree of similarity that is a sum of distances calculated for various items, and extract similar patients from the plurality of past patients on the basis of the calculated degree of similarity. The similar patient extraction function 62 may extract patients having highest degrees of similarity among the plurality of past patients as similar patients.

The similar patient extraction function 62 may access a patient information database that stores medical images of a plurality of patients. The similar patient extraction function 62 may further make an evaluation of a similarity measure for the plurality of patients based on image analysis of the obtained accompanying information and the medical images of the plurality of patients. The similar patient extraction function 62 may further extract similar patient similar to the target patient using the evaluation of the similarity measure.

The reference medical information obtaining function 63 obtains a plurality of medical documents, that is, text information, created for the similar patients extracted by the similar patient extraction function 62. The reference medical information obtaining function 63 obtains a medical document of each of the plurality of similar patients received from the electronic medical record system or the RIS through, for example, communication with the electronic medical record system or the RIS via the communication interface 4. The reference medical information obtaining function 63 may read and obtain, from the memory 5, the medical document of each of the plurality of similar patients received from the electronic medical record system or the RIS and stored in the memory 5.

The reference medical information obtaining function 63 may further obtain a plurality of medical images created for the similar patients extracted by the similar patient extraction function 62.

The medical document creation function 64 creates a medical document of the target patient (i.e., a document regarding a medical image of the target patient) on the basis of the accompanying information regarding the target patient obtained by the accompanying information obtaining function 61 and a summary of the plurality of medical documents of the similar patients obtained by the reference medical information obtaining function 63. The summary of the medical documents includes, for example, items and styles (that is, expressions) common among the plurality of medical documents created for each of the plurality of similar patients.

The medical document creation function 64 may create the medical document of the target patient using, for example, a large language model (LLM) such as ChatGPT. For example, the medical document creation function 64 may create the medical model of the target patient by inputting the accompanying information regarding the target patient and the medical documents of the plurality of similar patients to the LLM and causing the LLM to extract items and styles common to the input accompanying information regarding the target patient and the medical documents of the plurality of similar patients. In addition, the medical document creation function 64 may create the medical document of the target patient on the basis of a summary of two or more medical documents having a difference in content of a certain amount or more among the medical documents of the plurality of similar patients so that medical documents of similar patients having similar content are not used redundantly. In this case, the medical document creation function 64 may determine that a plurality of medical documents have a difference in content of the certain amount or more on the basis of degrees of similarity calculated on the basis of classical natural language processing such as term frequency-inverse document frequency (TF-IDF) and cosine similarity.

In addition, the medical document creation function 64 may further create a medical image of the target patient on the basis of the accompanying information regarding the target patient obtained by the accompanying information obtaining function 61 and the summary of the plurality of medical images of the similar patients obtained by the reference medical information obtaining function 63. The summary of the medical images may include an image area or the like common among the plurality of medical images created for each of the plurality of similar patients. The medical document creation function 64 may obtain the summary of the plurality of medical images created for the similar patients by analyzing the medical images.

In addition, the medical document creation function 64 may create a plurality of medical documents as the medical document of the target patient instead of one medical document. When the medical document creation function 64 creates a plurality of medical documents, the operator may select a desired medical document from the plurality of created medical documents.

The display control function 65 displays the medical document of the target patient created by the medical document creation function 64. The display control function 65 may further display a medical image corresponding to the medical document of the target patient.

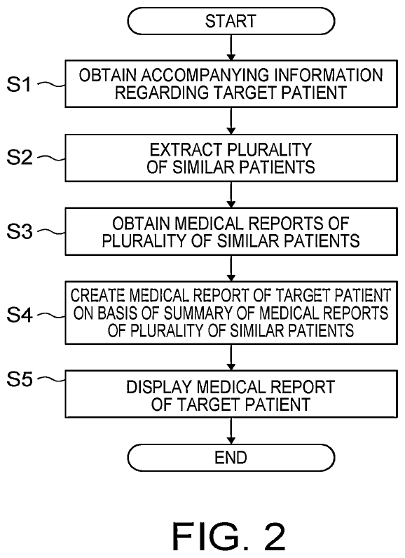

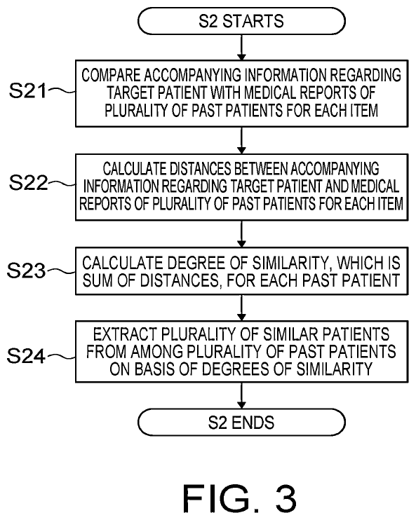

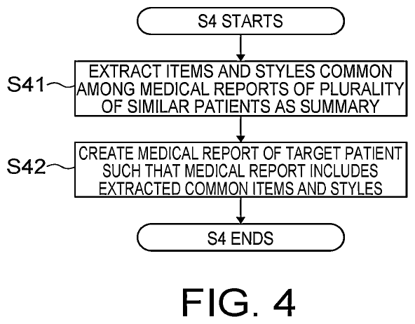

Next, an operation example of the medical information processing apparatus 1 according to the embodiment configured as described above will be described. FIG. 2 is a flowchart illustrating an operation example of the medical information processing apparatus 1 according to the embodiment. FIG. 3 is a flowchart illustrating a step of extracting similar patients in the operation example of the medical information processing apparatus 1 according to the embodiment. FIG. 4 is a flowchart illustrating a step of creating a medical report of a target patient in the operation example of the medical information processing apparatus 1 according to the embodiment.

As illustrated in FIG. 2, first, the accompanying information obtaining function 61 obtains accompanying information regarding the target patient. Specifically, the accompanying information obtaining function 61 obtains the accompanying information received from the electronic medical record system or the RIS via the communication interface 4 when the input interface 2 receives an input operation by the operator for obtaining accompanying information regarding the target patient. For example, when the operator inputs an identification number of the target patient, the accompanying information obtaining function 61 may obtain accompanying information regarding the target patient associated with the input identification number from the electronic medical record system or the RIS. Alternatively, the accompanying information obtaining function 61 may obtain accompanying information regarding the target patient manually input by the operator via the input interface 2. The accompanying information regarding the target patient includes, for example, at least one of an examination purpose, a patient attribute, a medical image, information regarding an image area of interest, and an examination finding. These pieces of accompanying information may be obtained in the form of examination order data, image data, patient attribute data, medical care phase information, and the like. Information regarding the image area of interest and the examination finding may be manually input by the operator.

After the accompanying information regarding the target patient is obtained, the similar patient extraction function 62 extracts a plurality of similar patients on the basis of the obtained accompanying information (step S2). Specifically, as illustrated in FIG. 3, first, the similar patient extraction function 62 compares the accompanying information regarding the target patient with medical reports of a plurality of past patients for each item (step S21). The medical report includes a medical document and a medical image corresponding to the medical document. The medical report is, for example, an interpretation report. The similar patient extraction function 62 receives and obtains the medical reports of the plurality of past patients from the electronic medical record system or the RIS through, for example, communication with the electronic medical record system or the RIS via the communication interface 4. In the medical reports of the plurality of past patients, at least one of an examination purpose, a patient attribute, a medical image, information regarding an image area of interest, and an examination finding is included as an item. Note that the image area of interest may be specified by attaching text information (for example, “a nodule is observed in the right lung” or the like) indicating the image area of interest to an image as a hyperlink. The examination finding may be associated with the hyperlink attached to the image.

Next, the similar patient extraction function 62 calculates, for each item, a distance between the accompanying information regarding the target patient and the medical reports of the plurality of past patients. In a case where the item is “finding”, for example, the similar patient extraction function 62 calculates a shorter distance as the number of matches between a plurality of keywords constituting the finding of the target patient and a plurality of keywords constituting the finding of each past patient becomes larger. Furthermore, in a case where the item is “image area of interest”, for example, the similar patient extraction function 62 calculates a shorter distance as anatomical positions and structures of the image area of interest of the target patient and the image area of interest of each past patient become closer to each other. Furthermore, in a case where the item is “examination purpose”, for example, the similar patient extraction function 62 calculates a shorter distance when the examination purposes of the target patient and each past patient are the same than when the examination purposes of the target patient and each past patient are different from each other. Furthermore, in a case where the item is “patient attribute” such as gender and age of the patient, for example, the similar patient extraction function 62 calculates a shorter distance as the patient attribute of the target patient and the patient attribute of each past patient become closer to each other. Furthermore, in a case where the item is “disease” or “medical care phase”, for example, the similar patient extraction function 62 calculates a shorter distance when the diseases and the medical care phases of the target patient and each past patient are the same than when the diseases and the medical care phases of the target patient and each past patient are different from each other.

After calculating the distance for each item, the similar patient extraction function 62 calculates, for each past patient, a degree of similarity, which is the sum of the distances for the items (step S23). A past patient having a smaller sum of distances has a higher degree of similarity to the target patient.

After calculating the degrees of similarity, the similar patient extraction function 62 extracts similar patients from the plurality of past patients on the basis of the calculated degrees of similarity (step S24). Specifically, the similar patient extraction function 62 extracts a plurality of past patients with high degrees of similarity as similar patients.

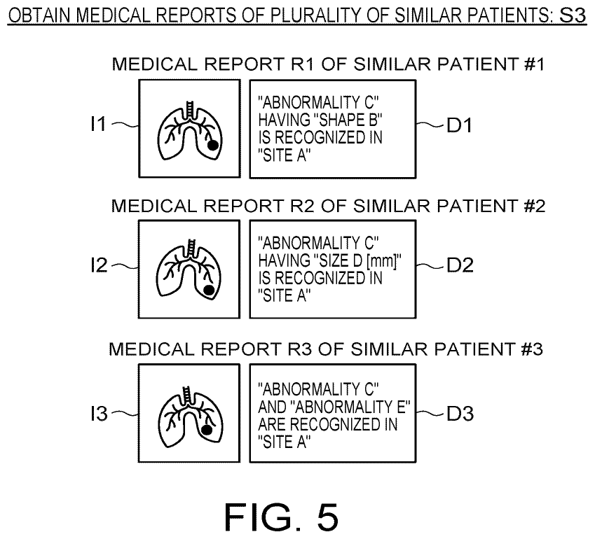

After the plurality of similar patients is extracted, the reference medical information obtaining function 63 obtains medical reports of the plurality of similar patients as illustrated in FIG. 2 (step S3). FIG. 5 is a diagram illustrating a step of obtaining medical reports of a plurality of similar patients in the operation example of the medical information processing apparatus 1 according to the embodiment. In the example illustrated in FIG. 5, the reference medical information obtaining function 63 obtains medical reports R1 to R3 of a plurality of similar patients #1 to #3 extracted by the similar patient extraction function 62. In the example illustrated in FIG. 2, the medical reports R1 to R3 of the plurality of similar patients #1 to #3 are stored in the memory 5 as part of the medical reports of the plurality of past patients when the similar patients are extracted. In the example illustrated in FIG. 5, the medical reports R1 to R3 of the plurality of similar patients #1 to #3 include medical documents D1 to D3 (that is, findings) and medical images I1 to I3 (that is, key images).

After the medical reports of the plurality of similar patients are obtained, the medical document creation function 64 creates a medical report of the target patient on the basis of a summary of the medical reports of the plurality of similar patients as illustrated in FIG. 2 (step S4).

Specifically, as illustrated in FIG. 4, first, the medical document creation function 64 extracts items and styles common among the medical reports of the plurality of similar patients as a summary (step S41). In the example illustrated in FIG. 5, the medical document creation function 64 extracts “site A” and “abnormality C” as items common to the medical reports R1 to R3 of the plurality of similar patients #1 to #3, and extracts “ . . . recognized” and the like as common styles.

After extracting items and styles common among the medical reports of the plurality of similar patients as a summary, the medical document creation function 64 creates a medical report of the target patient in such a way as to include the extracted common items and styles (step S42).

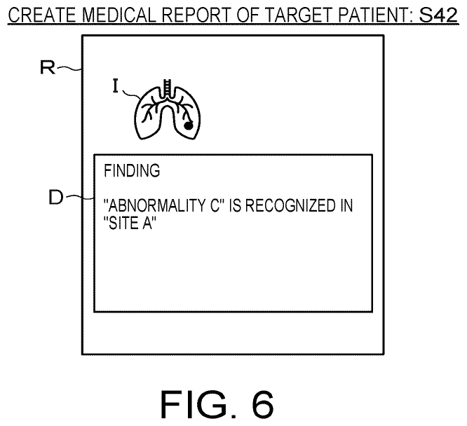

FIG. 6 is a diagram illustrating the step of creating a medical report of a target patient in the operation example of the medical information processing apparatus 1 according to the embodiment. In the example illustrated in FIG. 6, the medical document creation function 64 creates a medical report R of the target patient including a medical document D and a medical image I including the items and the styles common to the medical reports R1 to R3 of the plurality of similar patients #1 to #3.

After the medical report of the target patient is created, the display control function 65 displays the created medical report of the target patient as illustrated in FIG. 2 (step S5).

As described above, in the present embodiment, the accompanying information obtaining function 61 obtains the accompanying information regarding the target patient. In addition, the similar patient extraction function 62 extracts similar patients, who are similar to the target patient, on the basis of the accompanying information obtained by the accompanying information obtaining function 61. In addition, the reference medical information obtaining function 63 obtains a plurality of medical documents created for the similar patients extracted by the similar patient extraction function 62. In addition, the medical document creation function 64 creates the medical document of the target patient on the basis of the accompanying information obtained by the accompanying information obtaining function 61 and the summary of the plurality of medical documents obtained by the reference medical information obtaining function 63.

As a result, it is possible to create a medical document of a target patient on the basis of a summary of a plurality of medical documents created for similar patients, who are similar to the target patient, and it is therefore possible to support creation of a convenient and appropriate medical document.

In the present embodiment, the display control function 65 displays the medical document of the target patient created by the medical document creation function 64.

As a result, convenience can be improved.

Furthermore, in the present embodiment, the medical document creation function 64 creates a medical document of a target patient using an LLM.

This makes it possible to support creation of a more convenient and appropriate medical document.

Furthermore, in the present embodiment, the similar patient extraction function 62 compares accompanying information of a target patient with a plurality of medical documents created for each of a plurality of past patients for each of a plurality of items, and calculates distances between the accompanying information and the plurality of medical documents for each item. Furthermore, the similar patient extraction function 62 calculates degrees of similarity, each of which is the sum of distances calculated for the items, and extracts similar patients from among the plurality of past patients on the basis of the calculated degrees of similarity.

This makes it possible to easily and appropriately extract similar patients.

Furthermore, in the embodiment, the similar patient extraction function 62 extracts patient with highest degrees of similarity among the plurality of past patients as similar patients.

This makes it possible to more appropriately extract similar patients.

Furthermore, in the embodiment, the medical document creation function 64 may create a medical document of a target patient on the basis of a summary of two or more medical documents having a difference in content of a certain amount or more among the medical documents of the plurality of similar patients.

As a result, it is possible to prevent content of the medical document of the target patient from being biased to content of medical documents of specific past similar patients, and it is therefore possible to support creation of a more appropriate medical document.

Furthermore, in the embodiment, a summary of medical documents includes items and styles that are common among a plurality of medical documents created for each of a plurality of similar patients.

As a result, it is possible to support creation of a more appropriate medical document on the basis of items and styles common among the medical documents of the plurality of similar patients.

Furthermore, in the embodiment, the reference medical information obtaining function 63 further obtains medical images created for the similar patients extracted by the similar patient extraction function 62. Furthermore, the medical document creation function 64 further creates a medical image of the target patient on the basis of the accompanying information obtained by the accompanying information obtaining function 61 and the summary of the medical images obtained by the reference medical information obtaining function 63.

This makes it possible to support creation of a convenient and appropriate medical image.

Furthermore, in the embodiment, the accompanying information regarding the target patient includes at least one of an examination purpose, a patient attribute, a medical image, information regarding an image area of interest, and an examination finding.

This makes it possible to support creation of a more convenient and appropriate medical document.

### First Modification

Next, a first modification, in which medical documents and medical images created for similar patients are further displayed, will be described while focusing on differences from the above-described embodiment.

In the first modification, when displaying a medical document and a medical image of a target patient, the display control function 65 further displays medical documents created for similar patients and medical images created for similar patients.

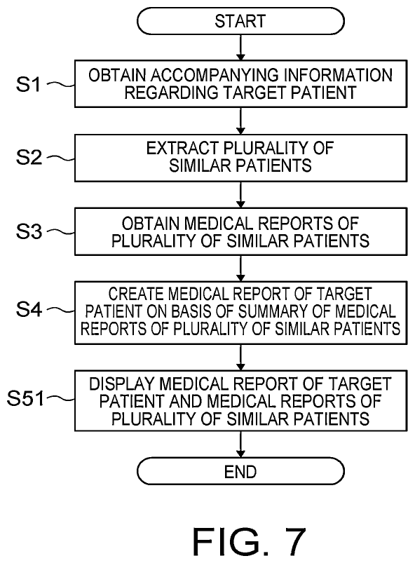

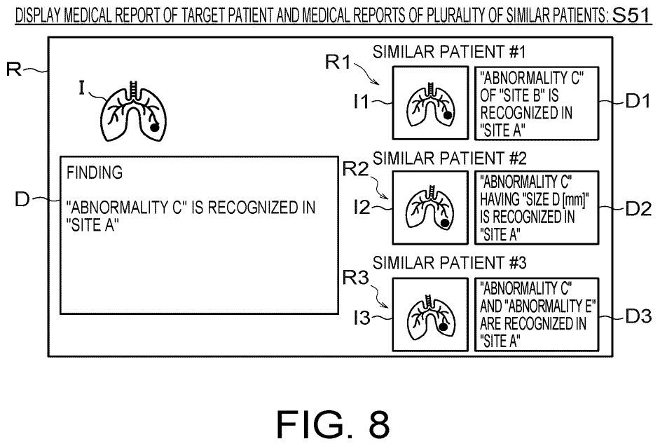

FIG. 7 is a flowchart illustrating an operation example of a medical information processing apparatus 1 according to the first modification of the embodiment. FIG. 8 is a diagram illustrating a step of displaying a medical report of a target patient and medical reports of a plurality of similar patients in the operation example of the medical information processing apparatus 1 according to the first modification of the embodiment. In the examples illustrated in FIGS. 7 and 8, after the medical document creation function 64 creates the medical report of the target patient, the display control function 65 displays a medical report R of the target patient and medical reports R1 to R3 of similar patients #1 to #3 (step S51).

As described above, in the first modification, the display control function 65 further displays medical documents created for similar patients and medical images created for the similar patients.

As a result, since the operator can easily understand that the medical document and the medical image of the target patient are created on the basis of the medical documents and the medical images of the similar patients, credibility of support for creation of a medical document and a medical image can be improved.

### Second Modification

Next, a second modification, in which a medical document of a target patient is corrected in accordance with an input operation, will be described while focusing on differences from the above-described embodiment.

In the second modification, the input interface 2 receives an input operation for correcting (that is, updating) a medical document of a target patient displayed by the display control function 65. The medical document creation function 64 corrects the medical document of the target patient in accordance with the input operation. The input interface 2 may receive an input operation for correcting a medical image of a target patient displayed by the display control function 65. The medical document creation function 64 may correct the medical image of the target patient in response to the input operation.

Specifically, in the second modification, the medical document creation function 64 creates, as a medical document of a target patient, a medical document in which items and styles common among medical documents of a plurality of similar patients are included and specific information can be added, through input operations, for items having differences between the medical documents of the plurality of similar patients. Furthermore, in the second modification, the similar patient extraction function 62 narrows the number of similar patients in accordance with input operation. Furthermore, in the second modification, the reference medical information obtaining function 63 narrows the number of medical documents of similar patients in accordance with the narrowing down of the number of similar patients by the similar patient extraction function 62. Furthermore, in the second modification, the medical document creation function 64 corrects the medical document of the target patient on the basis of a summary of medical documents remaining after the narrowing down by the reference medical information obtaining function 63.

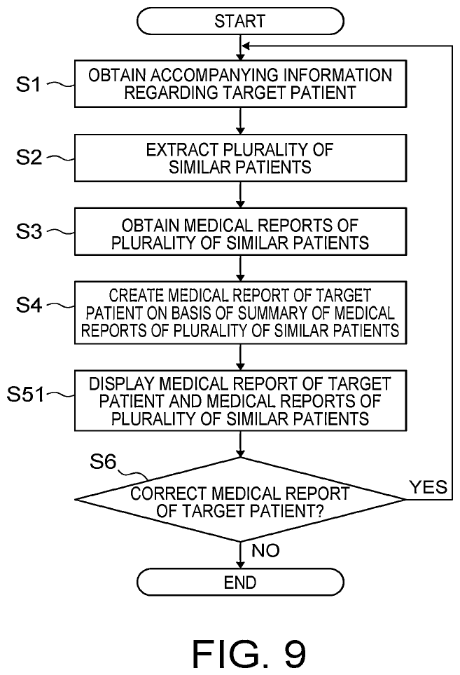

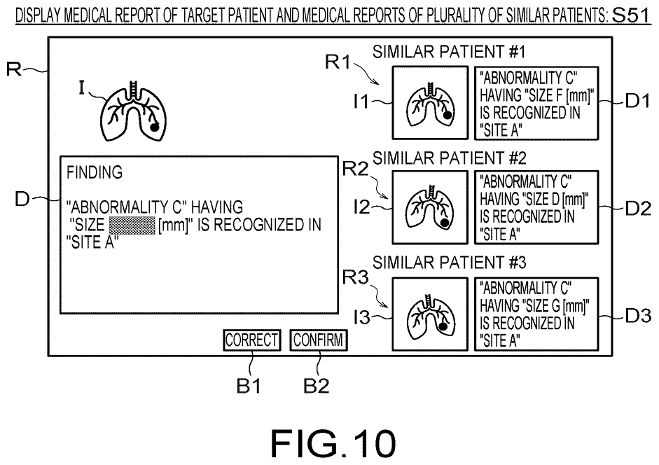

FIG. 9 is a flowchart illustrating an operation example of a medical information processing apparatus 1 according to the second modification of the embodiment. FIG. 10 is a diagram illustrating a step of displaying a medical report of a target patient and medical reports of a plurality of similar patients in the operation example of the medical information processing apparatus 1 according to the second modification of the embodiment.

In the example illustrated in FIG. 10, the medical document creation function 64 generates a medical report R of the target patient in a state where a specific value [mm] of “size” of an abnormality C, which is an item having a difference between medical reports of a plurality of similar patients, can be added through an input operation. That is, the medical document creation function 64 generates the medical report R of the target patient that can be filled with a specific value of the size of the abnormality C through an input operation. Furthermore, in the example illustrated in FIG. 10, the medical document creation function 64 generates a medical report R of the target patient including a correct button B1 that receives an input operation for correcting content of the medical report R of the target patient and a confirm button B2 that receives an input operation for confirming the content of the medical report R of the target patient. When the correct button B1 is selected, an input operation for inputting a specific value of the size of the abnormality C is received.

As illustrated in FIG. 9, if the input interface 2 receives an input operation for correcting the medical report R of the target patient (step S6: YES), the accompanying information obtaining function 61 obtains input information based on the input operation as accompanying information regarding the target patient (step S1). That is, in the example illustrated in FIG. 9, the accompanying information regarding the target patient includes the input information based on the input operation for correcting the medical report R of the target patient. In the example illustrated in FIG. 10, the accompanying information obtaining function 61 obtains a specific value of the size of the abnormality C input through the input operation as the accompanying information regarding the target patient. Next, the similar patient extraction function 62 narrows the number of similar patients in accordance with an input operation (step S2). In the example illustrated in FIG. 10, the similar patient extraction function 62 extracts similar patients whose medical reports include the specific value of the size of the abnormality C input through the input operation from the plurality of similar patients already extracted, thereby narrowing down the number of similar patients. Next, the reference medical information obtaining function 63 narrows down the number of medical reports of similar patients in accordance with the narrowing down of the number of similar patients by the similar patient extraction function 62 (step S3). That is, the reference medical information obtaining function 63 obtains medical reports of similar patients remaining after the narrowing down. Next, the medical document creation function 64 corrects, that is, re-creates, the medical report of the target patient on the basis of a summary of the medical reports remaining after the narrowing down by the reference medical information obtaining function 63 (step S4).

If the input interface 2 does not receive an input operation for correcting the medical report R of the target patient (step S6: No), on the other hand, the process ends. Note that, if the similar patient extraction function 62 can extract a similar patient with a very high degree of similarity described above, there is a high possibility that a medical report of the similar patient can be used as the medical report of the target patient as is. A threshold, therefore, is set for the degree of similarity, and if a degree of similarity exceeds the threshold, a medical report of a corresponding patient may be created as the medical report of the target patient as is, and the process may end without accepting the correction.

As described above, in the second modification, the input interface 2 receives an input operation for correcting the medical document of the target patient displayed by the display control function 65. Furthermore, the medical document creation function 64 corrects the medical document of the target patient in accordance with an input operation.

As a result, it is possible to support creation of a more appropriate medical document by receiving an input operation for correcting a medical document of a target patient.

Furthermore, in the second modification, the accompanying information regarding the target patient includes input information based on an input operation for correcting the medical document of the target patient. Furthermore, the similar patient extraction function 62 narrows down the number of similar patients in accordance with the input operation for correcting the medical document. That is, the similar patient extraction function 62 narrows the number of similar patients as the accompanying information obtaining function 61 obtains, as the accompanying information, the input information based on the input operation for correcting the medical document of the target patient. Furthermore, the reference medical information obtaining function 63 narrows down the number of medical documents in accordance with the narrowing down of the number of similar patients by the similar patient extraction function 62. Furthermore, the medical document creation function 64 corrects the medical document of the target patient on the basis of a summary of medical documents remaining after the narrowing down by the reference medical information obtaining function 63.

As a result, it is possible to bring the medical document of the target patient closer to completion in accordance with progress of input operations for correcting the medical document of the target patient, and it is therefore possible to support creation of a more convenient and appropriate medical document.

Furthermore, in the second modification, the medical document creation function 64 creates, as a medical document of a target patient, a medical document in which items and styles common among medical documents of a plurality of similar patients are included and specific information can be added, through input operations, for items having differences between the medical documents of the plurality of similar patients.

This makes it possible to support creation of a more convenient and appropriate medical document.

### Third Modification

Next, a third modification, in which a medical document of a target patient is corrected in accordance with an input operation, will be described while focusing on differences from the above-described embodiment.

In the second modification, the medical document creation function 64 creates, as a medical document of a target patient, a medical document in which items and styles common among medical documents of a plurality of similar patients are included and specific information can be added, through input operations, for items having differences between the medical documents of the plurality of similar patients. In the third modification, on the other hand, the medical document creation function 64 creates a medical document in which any of options can be selected, through input operations, for some items having differences between medical documents of a plurality of similar patients.

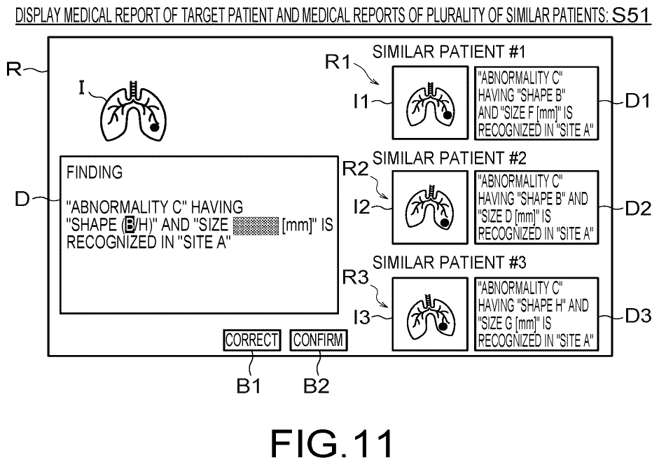

FIG. 11 is a diagram illustrating a step of displaying a medical report of a target patient and medical reports of a plurality of similar patients in an operation example of a medical information processing apparatus 1 according to the third modification of the embodiment. In the example illustrated in FIG. 11, the medical document creation function 64 generates the medical report R of the target patient in a state where any of options “B” and “H” can be selected, through an input operation, for “shape” of the abnormality C, which is an item having a difference between medical reports of similar patients. Note that, as in the second modification, a specific value can be input for “size” of the abnormality C through an input operation. When the correct button B1 is selected in FIG. 11, an input operation for selecting the shape of the abnormality C is received. When the input operation for selecting the shape of the abnormality C is received, the accompanying information obtaining function 61 obtains the selected shape as the accompanying information regarding the target patient. In response to this, the similar patient extraction function 62 narrows down the number of similar patients, and the reference medical information obtaining function 63 narrows down the number of medical documents of similar patients. The medical document creation function 64 then corrects the medical document of the target patient on the basis of a summary of medical documents remaining after the narrowing down by the reference medical information obtaining function 63.

As described above, in the third modification, the medical document creation function 64 creates, as a medical document of a target patient, a medical document in which items and styles common among medical documents of a plurality of similar patients are included and any of options can be selected, through input operations, for items having differences between the medical documents of the plurality of similar patients.

This makes it possible to support creation of a more convenient medical document.

### Fourth Modification

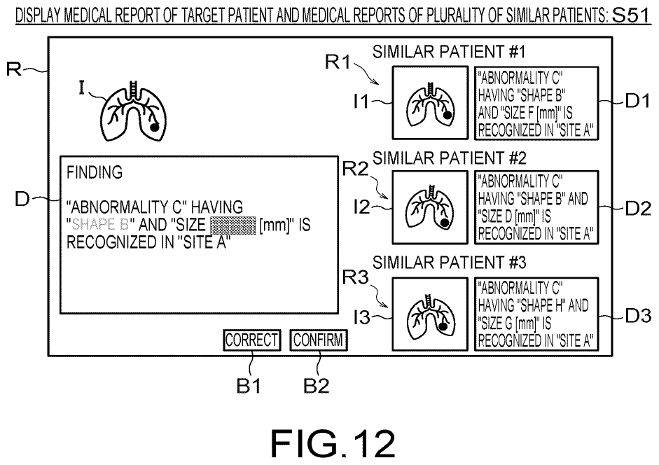

FIG. 12 is a diagram illustrating a step of displaying a medical report of a target patient and medical reports of a plurality of similar patients in an operation example of a medical information processing apparatus 1 according to a fourth modification of the embodiment.

In the fourth modification, the medical document creation function 64 creates, as a medical document of a target patient, a medical document in which items having differences between medical documents of a plurality of similar patients, the differences being smaller than those of items for which specific information can be added or any of options can be selected through input operations, are described in character colors corresponding to magnitude of the differences.

In an example illustrated in FIG. 12, the medical document creation function 64 creates, as a medical document of a target patient, a medical document in which “shape B”, which is an item having a smaller difference than “size” between medical reports of similar patients, is described in a character color different from that of “site A” or “abnormality C”, which is an item common between the medical reports of the similar patients. The character color of the item varies depending on magnitude of the difference between the medical reports of the similar patients. For example, the character color of the item is different between when the difference is medium and when the difference is small. “Shape B” may be correctable through selection of the correct button B1.

As described above, in the fourth modification, the medical document creation function 64 creates, as a medical document of a target patient, a medical document in which items having differences between a plurality of medical documents, the differences being smaller than those of items for which specific information can be added or any of options can be selected through input operations, are described in character colors corresponding to magnitude of the differences.

This can support creation of a more appropriate medical document.

### Fifth Modification

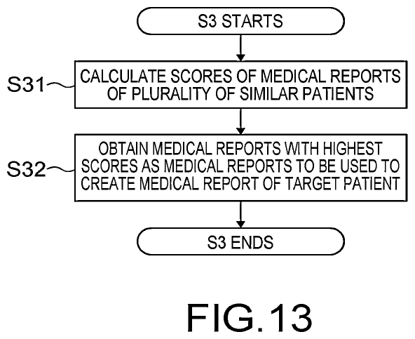

FIG. 13 is a flowchart illustrating a step of obtaining medical reports of similar patients in operation of a medical information processing apparatus 1 according to a fifth modification of the embodiment. As described above, by using medical documents of similar patients, it is possible to more conveniently and appropriately create a medical document of a target patient than before. Medical documents of similar patients obtained by the reference medical information obtaining function 63, however, include a wide variety of medical documents. That is, medical documents of similar patients obtained by the reference medical information obtaining function 63 do not necessarily have high utility value for creating a medical document of a target patient. In order to improve quality of a medical document of a target patient, it is desirable to obtain medical documents of similar patients having high utility value.

In an example illustrated in FIG. 13, therefore, the reference medical information obtaining function 63 obtains medical documents having high utility value among medical documents of a large number of similar patients as medical documents to be used to create a medical document of a target patient. Specifically, in the example illustrated in FIG. 13, first, the reference medical information obtaining function 63 calculates a score of each of medical reports of a plurality of similar patients (step S31). The score is a value indicating utility value of each medical report. The score may be a value of 0 or more and 100 or less. The reference medical information obtaining function 63 may calculate a score on the basis of a preset index related to calculation of a score.

The index related to the calculation of a score may be a record of use of each medical report. That is, the reference medical information obtaining function 63 may calculate a high score as a record of use of each medical report becomes better. The record of use of each medical report may include a plurality of items (that is, subdivided indices) such as the number of times that the medical report has been referred to and presence or absence of a record of use of the medical report in a conference system. In this case, the reference medical information obtaining function 63 may calculate a cost at which a value increases stepwise in accordance with an increase in the number of times that each medical report is referred to. Furthermore, when there is a record of use of each medical report in the conference system, the reference medical information obtaining function 63 may calculate a higher score than when there is no record.

Alternatively, the index related to the calculation of a score may be man-hours required to create each medical report. That is, the reference medical information obtaining function 63 may calculate a higher score as the number of man-hours required to create each medical report increases. The man-hours required to create each medical report may include a plurality of items such as whether or not a medical image is attached to the medical report as a key image and the number of characters in the medical report. In this case, when a medical image is attached to the medical report as a key image, the reference medical information obtaining function 63 may calculate a higher score than when no medical image is attached. Furthermore, the reference medical information obtaining function 63 may calculate a cost at which a value increases stepwise in accordance with an increase in the number of characters of each medical report.

Alternatively, the index related to the calculation of a score may be credibility of content of each medical report. That is, the reference medical information obtaining function 63 may calculate a higher score as the credibility of content of each medical report becomes higher. The credibility of content of each medical report may be lowered when a finding having content different from the content of the medical report for which a score is to be calculated is attached to another medical report of the same patient. When there is a finding that “abnormality A is recognized” in a medical report for which a score is to be calculated but there is a finding that “abnormality A is not recognized” in another medical report of the same patient, for example, credibility of content of the medical report may be lowered. In this case, when a finding having content different from content of a medical report for which a score is to be calculated is attached to another medical report of the same patient, the reference medical information obtaining function 63 may calculate a lower score than when the finding is not attached.

In addition to the above, the index related to the calculation of a score may be proficiency of a creator of each medical report (that is, years of experience of a doctor). That is, the reference medical information obtaining function 63 may calculate a higher score as proficiency of a creator of each medical report becomes higher. In this case, the reference medical information obtaining function 63 may calculate a score according to the proficiency of the creator by referring to a score table in which information regarding a creator of the medical report is associated with the score according to the proficiency of the creator of the medical report.

The reference medical information obtaining function 63 may obtain information regarding the index for calculating the score of the medical report together with medical reports of similar patients from the electronic medical record system or the RIS. The information regarding the index is, for example, the number of times that the medical report has been referred to, presence or absence of a record of use of the medical report in the conference system, whether or not a medical image is attached to the medical report as a key image, the number of characters of the medical report, different findings in other medical reports of the same patient, information regarding a creator of the medical report, or the like as described above. The reference medical information obtaining function 63 may then calculate a score based on the index using the obtained information regarding the index.

After calculating the score, the reference medical information obtaining function 63 obtains, on the basis of the calculated scores, medical reports having highest scores among the medical reports of the plurality of similar patients as medical reports to be used to create a medical report of the target patient (step S32). For example, the reference medical information obtaining function 63 obtains medical reports of similar patients having scores of 80 or more as medical reports to be used to create the medical report of the target patient.

Note that the reference medical information obtaining function 63 may calculate, on the basis of a plurality of indices regarding scores, scores of a plurality of medical reports for each of the indices. The plurality of indices may include at least two of a record of use of the medical report, the man-hours required to create the medical report, credibility of content of the medical report, and proficiency of a creator of the medical report. In this case, the reference medical information obtaining function 63 combines calculated scores for the indices to calculate a final score of each of the plurality of medical reports. The combination of the scores for the indices is, for example, simply summing (that is, addition) the scores for the indices. Alternatively, the combination of the scores for the indices may be calculating an average of the scores for the indices. Alternatively, the combination of the scores for the indices may be a sum of values obtained by multiplying the scores for the indices by weighting coefficients corresponding to the indices. The reference medical information obtaining function 63 then obtains medical documents to be used to create the medical document of the target patient from among the plurality of medical documents on the basis of the calculated final scores. For example, the reference medical information obtaining function 63 obtains medical reports of similar patients having final scores equal to or higher than a threshold as medical reports to be used to create the medical report of the target patient.

As described above, in the fifth modification, the reference medical information obtaining function 63 calculates a score of each of a plurality of medical documents created for similar patients and obtains medical documents to be used to create a medical document of a target patient from among the plurality of medical documents on the basis of calculated scores.

As a result, medical documents having high utility value can be obtained, and quality of a medical document of a target patient can be improved.

Furthermore, in the fifth modification, the reference medical information obtaining function 63 can calculate a final score of each of a plurality of medical documents by calculating scores of the plurality of medical documents for a plurality of indices related to the calculation of scores on the basis of the indices and combining the calculated scores for the indices. The reference medical information obtaining function 63 can then obtain medical documents to be used to create the medical document of the target patient from among the plurality of medical documents on the basis of the calculated final scores.

As a result, a medical document having high utility value can be more appropriately obtained, and quality of the medical document of the target patient can be further improved.

Note that a term “processor” used in the above description means, for example, a central processing unit (CPU), a graphics processing unit (GPU), or a circuit such as an application-specific integrated circuit (ASIC) or a programmable logic device (for example, a simple programmable logic device (SPLD), a complex programmable logic device (CPLD), or a field-programmable gate array (FPGA)). The processor achieves functions by reading and executing programs stored in the storage circuitry. Note that, instead of storing the programs in the storage circuitry, the programs may be directly incorporated in the circuitry of the processor. In this case, the processor achieves the functions by reading and executing the programs incorporated in the circuitry. Note that the processor is not limited to a case where the processor is configured as a single processor circuit, and a plurality of independent circuits may be combined to be configured as one processor to achieve the functions. Furthermore, the plurality of components in FIG. 1 may be integrated into one processor to achieve the functions.

According to at least one embodiment described above, it is possible to support creation of a convenient and appropriate medical document.

Although several embodiments have been described above, these embodiments have been presented only as examples, and are not intended to limit the scope of the invention. The novel apparatus and method described herein can be implemented in various other modes. In addition, various omissions, substitutions, and changes can be made to the modes of the apparatus and the method described herein without departing from the gist of the invention. The appended claims and their equivalents are intended to include such modes and modifications included in the scope and gist of the invention.

What is claimed is:

1. A medical image analyzing apparatus comprising:
processing circuitry configured to:
obtain a medical image of a target patient and information indicating an area of interest in the medical image as accompanying information;
access a patient information database that stores medical images of a plurality of patients, make an evaluation of a similarity measure for the plurality of patients based on image analysis of the obtained accompanying information and the medical images of the plurality of patients, and extract similar patient similar to the target patient using the evaluation of the similarity measure;
obtain a plurality of medical images and a plurality of medical documents created for the extracted similar patients; and
obtain a summary of the plurality of medical images by analyzing the medical images, and create a document regarding the medical image of the target patient based on the summary of the medical images, the obtained accompanying information, and a summary of the plurality of obtained medical documents.

2. The medical image analyzing apparatus according to claim 1, wherein the processing circuitry is further configured to display the created document regarding the medical image of the target patient.

3. The medical image analyzing apparatus according to claim 1, wherein the processing circuitry is further configured to create the document regarding the medical image of the target patient using a large language model.

4. The medical image analyzing apparatus according to claim 1, wherein the processing circuitry is further configured, in addition to extract the similar patients using the evaluation of the similarity measure based on the image analysis, to compare the accompanying information regarding the target patient with a plurality of medical documents created for each of a plurality of past patients for each of a plurality of items, calculate distances between the accompanying information and the plurality of medical documents for each of the items, calculate degrees of similarity that are sums of the distances calculated for the items, and extract the similar patients from the plurality of past patients based on the calculated degrees of similarity.

5. The medical image analyzing apparatus according to claim 4, wherein the processing circuitry is further configured to extract patients having at least one of highest similarity measure and highest degrees of similarity among the plurality of past patients as the similar patients.

6. The medical image analyzing apparatus according to claim 1, wherein
the processing circuitry is further configured to:
extract a plurality of similar patients;
obtain a plurality of medical documents created for each of the plurality of similar patients; and
create the document regarding the medical image of the target patient based on a summary of two or more medical documents having a difference in content of a certain amount or more among the plurality of medical documents.

7. The medical image analyzing apparatus according to claim 2, further comprising:
an input interface configured to receive an input operation for correcting the displayed document regarding the medical image of the target patient, wherein
the processing circuitry is further configured to correct the document regarding the medical image of the target patient in accordance with the input operation.

8. The medical image analyzing apparatus according to claim 7, wherein
the processing circuitry is further configured to:
narrow down a number of similar patients in accordance with the input operation;
narrow down a number of medical images and medical documents in accordance with the narrowing down of the number of similar patients; and
correct the document regarding the medical image of the target patient based on a summary of medical images and a summary of medical documents remaining after the narrowing down of the number of medical images and medical documents.

9. The medical image analyzing apparatus according to claim 1, wherein
the processing circuitry is further configured to extract a plurality of similar patients, and
the summary of the medical documents includes items and styles common among a plurality of medical documents created for each of the plurality of similar patients.

10. The medical image analyzing apparatus according to claim 7, wherein
the processing circuitry is further configured to extract a plurality of similar patients,
the summary of the medical documents includes items and styles common among a plurality of medical documents created for each of the plurality of similar patients, and
the processing circuitry is further configured to create, as the document regarding the medical image of the target patient, a document in which an item and a style common among the plurality of medical documents are included and specific information is addable through the input operation or any of options is selectable through the input operation for an item having a difference among the plurality of medical documents.

11. The medical image analyzing apparatus according to claim 10, wherein the processing circuitry is further configured to create, as the document regarding the medical image of the target patient, a document in which an item having a difference between the plurality of medical documents, the difference being smaller than a difference of an item for which specific information is addable or any of options is selectable through the input operation, is described in a character color according to magnitude of the difference.

12. The medical image analyzing apparatus according to claim 2, wherein the processing circuitry is further configured to further display the plurality of medical documents created for the similar patients and a plurality of medical images created for the similar patients.

13. The medical image analyzing apparatus according to claim 1, wherein the processing circuitry is further configured to obtain at least one of an examination purpose, a patient attribute, and an examination finding as the accompanying information.

14. The medical image analyzing apparatus according to claim 7, wherein the processing circuitry is further configured to obtain, as the accompanying information, at least one of an examination purpose, a patient attribute, and an examination finding, and input information based on the input operation for correcting the medical document of the target patient.

15. The medical image analyzing apparatus according to claim 1, wherein the processing circuitry is further configured to calculate a score of each of the plurality of medical documents created for the similar patients and obtain, based on the calculated score, a medical document to be used to create the document regarding the medical image of the target patient from among the plurality of medical documents.

16. The medical image analyzing apparatus according to claim 15, wherein the processing circuitry is further configured to calculate, based on a plurality of indices related to calculation of scores, scores of the plurality of medical documents for each of the indices, combine the calculated scores for the indices to calculate a final score of each of the plurality of medical documents, and obtain, based on the calculated final score, a medical document to be used to create the document regarding the medical image of the target patient from among the plurality of medical documents.

17. The medical image analyzing apparatus according to claim 16, wherein the plurality of indices includes at least two of a record of use of a medical document, man-hours required to create the medical document, credibility of content of the medical document, and proficiency of a creator of the medical document.

18. A medical image analyzing method comprising: obtaining a medical image of a target patient and information indicating an area of interest in the medical image as accompanying information;
accessing a patient information database that stores medical images of a plurality of patients, making an evaluation of a similarity measure for the plurality of patients based on image analysis of the obtained accompanying information and the medical images of the plurality of patients, and extracting similar patient similar to the target patient using the evaluation of the similarity measure;
obtaining a plurality of medical images and a plurality of medical documents created for the extracted similar patients; and
obtaining a summary of the plurality of medical images by analyzing the medical images, and creating a document regarding the medical image of the target patient based on the summary of the medical images, the obtained accompanying information, and a summary of the plurality of obtained medical documents.

19. A medical information processing apparatus comprising:
processing circuitry configured to:
obtain accompanying information regarding a target patient;
extract similar patients, who are similar to the target patient, based on the obtained accompanying information;
obtain a plurality of medical documents created for the extracted similar patients; and
create a medical document of the target patient based on the obtained accompanying information and a summary of the plurality of obtained medical documents.
